(19) United States
(12) Patent Application Publication
(10) Pub. No.: US 2026/0276865 A1
(43) Pub. Date: Sep. 17, 2026

(54) INDEX-MATCHED, BULK-SCATTERING OPTICAL FIBERS FOR ENHANCING PHOTOSYNTHESIS BY PHOTOSYNTHETIC MICROORGANISMS AND TO METHODS OF PREPARING AND USING THE SAME

(71) Applicant: Board of trustees of Michigan State University, East Lansing, MI (US)

(72) Inventors: Xinyue LIU, East Lansing, MI (US); Yijie CHENG, East Lansing, MI (US)

(73) Assignee: Board of trustees of Michigan State University, East Lansing, MI (US)

(21) Appl. No.: 19/566,360

(22) Filed: Mar. 13, 2026

Related U.S. Application Data
(60) Provisional application No. 63/771,881, filed on Mar. 14, 2025.

(51) Int. Cl. G02B 1/04; B82Y 20/00; C12M 1/00

(52) U.S. Cl. CPC G02B 1/046; B82Y 20/00; C12M 21/02; C12M 31/10

(57) ABSTRACT

An index-matched, bulk scattering optical fiber for use during photosynthesis by a photosynthetic microorganism includes a hydrogel matrix and a plurality of nanoparticles dispersed in the hydrogel matrix. A weight fraction of the nanoparticles in the hydrogel matrix ranges from greater than 0 weight percent to less than or equal to about 0.36 weight percent. A method of using the optical fiber includes contacting the optical fiber and the photosynthetic microorganism. A photobioreactor for use during photosynthesis by a photosynthetic microorganism includes a housing that defines a cavity, the cavity being configured to receive a photosynthetic microorganism and the optical fiber.

### CROSS-REFERENCE TO RELATED APPLICATIONS

This application claims the benefit of U.S. Provisional Application No. 63/771,881, filed on Mar. 14, 2025. The entire disclosure of the above application is incorporated herein by reference.

### GOVERNMENT SUPPORT

This invention was made with government support under 2401017 and 2320716 awarded by the National Science Foundation. The government has certain rights in the invention.

### FIELD

The present disclosure relates to index-matched, bulk-scattering optical fibers for enhancing photosynthesis by photosynthetic microorganisms and to methods of preparing and using the same.

### BACKGROUND

This section provides background information related to the present disclosure which is not necessarily prior art.

Photosynthesis is a cornerstone of the global carbon cycle, enabling terrestrial plants and aquatic algae to convert solar energy into chemical energy, with an estimated annual carbon fixation of 240 billion tons. This biological process has inspired photosynthetic biomanufacturing, an emerging industry focused on harnessing solar energy and atmospheric carbon to produce biofuels, biopharmaceuticals, biomaterials, and value-added chemicals. Algae, in particular, are highly attractive biomanufacturing platforms due to their rapid growth, high photosynthetic efficiency, and adaptability to diverse environments. However, translating this potential into high-yield production systems remains limited by light delivery.

Algal photosynthesis is highly sensitive to local light availability. When light intensity falls below the compensation point, photosynthesis cannot exceed cell respiration, causing the algal cells to consume more energy than they produce, and in contrast, excessive light intensities can lead to oversaturation, where algal cells dissipate excess light as heat and fluorescence with photoinhibition behaviors. For example, productive photosynthesis for Chlorella sorokiniana typically occurs within a narrow light intensity range of 80 W·m⁻² to 220 W·m⁻². Intensifying this challenge, light is absorbed strongly by algal cells, creating steep spatial gradients in illumination across algal cultures. Even moderately dense cultures (e.g., 0.1 g·L⁻¹) can confine usable light to regions close to the surface, leaving most of the culture volume under-illuminated. These combined effects differ from conventional chemical or biochemical processes, where productivity generally increases with reactant concentration. In photosynthetic systems, increasing algal cell density may initially boost productivity but eventually leads to diminishing returns due to light limitation. Together, these observations highlight the need for strategies that provide more uniform light distribution throughout the culture volume to fully harness the photosynthetic capacity of algal systems.

To improve light penetration and algal growth in dense cultures, prior efforts have adopted artificial lighting systems, but these approaches generally suffer from high energy demand and poor conversion efficiency. For example, supplementing algal photobioreactors with light-emitting diode (LED) can deliver extremely high irradiance on the order of about 2,000 W·m⁻² to about 6,000 W·m⁻² but may require up to 5 MJ of electricity to produce just 1 gram of biomass, corresponding to a solar-to-biomass conversion efficiency of less than 0.3%. Thus, it is unsuitable for sustainable, large-scale bioproduction. Passive light modulation systems have also been explored, including waveguides made from transparent, high-refractive index polymers such as polymethyl methacrylate (PMMA), polycarbonate, or glass. These materials guide light via total internal reflection and typically require surface roughening to enable lateral emission. Nevertheless, lateral extraction remains inefficient due to refractive index mismatch and low photon-surface interactions, resulting in poor volumetric illumination and minimal enhancement of photosynthetic productivity.

### SUMMARY

This section provides a general summary of the disclosure, and is not a comprehensive disclosure of its full scope or all of its features.

At least one example embodiments relates to a method of using an index-matched, bulk-scattering optical fiber during photosynthesis by a photosynthetic microorganism.

In at least one example embodiment, the method may include contacting the optical fiber and the photosynthetic microorganism. The optical fiber may include a hydrogel matrix and a plurality of nanoparticles dispersed in the hydrogel matrix.

In at least one example embodiment, the hydrogel matrix may include a crosslinkable polymer. The crosslinkable polymer may include polyvinyl alcohol, poly(ethylene glycol), polyacrylamide, poly(acrylic acid), alginate, gelatin, chitosan, dextran, hyaluronic acid, cellulose derivatives, or any combination thereof.

In at least one example embodiment, the nanoparticles of the plurality of nanoparticles may include silica (SiO₂), zirconium dioxide (ZrO₂), titanium dioxide (TiO₂), zinc oxide (ZnO), aluminum oxide (Al₂O₃), or any combination thereof.

In at least one example embodiment, the nanoparticles of the plurality of nanoparticles may have a particle distribution greater than or equal to about 200 nanometers to less than or equal to about 1,800 nanometers.

In at least one example embodiment, a weight fraction of the plurality of nanoparticles in the hydrogel matrix may range from greater than 0 weight percent to less than or equal to about 0.36 weight percent.

In at least one example embodiment, the optical fiber may have an average diameter greater than or equal to about 0.4 millimeters to less than or equal to about 5 millimeters.

In at least one example embodiment, a relationship between the optical fiber and the photosynthetic microorganism may include greater than or equal to about 450 square centimeters to less than or equal to about 950 square centimeters of surface area of the optical fiber per about 1 gram of the photosynthetic microorganism.

In at least one example embodiment, the contacting may occur in a photobioreactor. The photobioreactor may include a housing that defines cavity configured to receive the photosynthetic microorganism and that supports the optical fiber.

In at least one example embodiment, the optical fiber may be a first optical fiber and the photobioreactor may further include a second optical fiber, where an average spacing between the first optical fiber and the second optical fiber may be greater than or equal to about 10 millimeters to less than or equal to about 20 millimeters.

In at least one example embodiment, the method may further include determining a first refractive-index of the photosynthetic microorganism and selecting the hydrogel matrix to have a second refractive-index that is the same as the first refractive-index.

In at least one example embodiment, the method may further include determining an optimal volumetric ratio of the optical fiber to the photosynthetic microorganism.

In at least one example embodiment, the method may further include preparing the hydrogel matrix. The preparing of the hydrogel matrix may include contacting a first hydrogel precursor solution including the nanoparticles and a second hydrogel precursor solution including a crosslinker. The first and second hydrogel precursor solutions may both include a crosslinkable polymer.

At least some example embodiments relate to an index-matched, bulk scattering optical fiber for use during photosynthesis by a photosynthetic microorganism.

In at least one example embodiment, the optical fiber may include a hydrogel matrix and a plurality of nanoparticles dispersed in the hydrogel matrix. A weight fraction of the nanoparticles in the hydrogel matrix may range from greater than 0 weight percent to less than or equal to about 0.36 weight percent.

In at least one example embodiment, the hydrogel matrix may include a crosslinkable polymer. The crosslinkable polymer may include polyvinyl alcohol, poly(ethylene glycol), polyacrylamide, poly(acrylic acid), alginate, gelatin, chitosan, dextran, hyaluronic acid, cellulose derivatives, or any combination thereof. The nanoparticles of the plurality of nanoparticles may include silica (SiO₂), zirconium dioxide (ZrO₂), titanium dioxide (TiO₂), zinc oxide (ZnO), aluminum oxide (Al₂O₃), or any combination thereof.

In at least one example embodiment, the nanoparticles of the plurality of nanoparticles may have a particle distribution greater than or equal to about 200 nanometers to less than or equal to about 1800 nanometers.

At least some example embodiments may include a photobioreactor for use during photosynthesis by a photosynthetic microorganism.

In at least one example embodiment, the photobioreactor may include a housing that defines a cavity, the cavity being configured to receive a photosynthetic microorganism and an optical fiber. The optical fiber may include a hydrogel matrix and a plurality of nanoparticles dispersed in the hydrogel matrix.

In at least one example embodiment, the hydrogel matrix may include a crosslinkable polymer. The crosslinkable polymer may include polyvinyl alcohol, poly(ethylene glycol), polyacrylamide, poly(acrylic acid), alginate, gelatin, chitosan, dextran, hyaluronic acid, cellulose derivatives, or any combination thereof. The nanoparticles of the plurality of nanoparticles may include silica (SiO₂), zirconium dioxide (ZrO₂), titanium dioxide (TiO₂), zinc oxide (ZnO), aluminum oxide (Al₂O₃), or any combination thereof.

In at least one example embodiment, the optical fiber may be a first optical fiber and the photobioreactor may further include one or more other optical fibers that are provided with the first optical fiber as an array of optical fibers.

In at least one example embodiment, the array of optical fibers may be configured to be received by the cavity and to be at least partially submerged into a bioreactor medium that includes the photosynthetic microorganism.

In at least one example embodiment, an average spacing between the first optical fiber and a second optical fiber of the one or more other optical fibers may be greater than or equal to about 10 millimeters to less than or equal to about 20 millimeters.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is an illustration of an example optical fiber in accordance with at least one example embodiment.

FIG. 2 is a sideview of an example photobioreactor including one or more optical fibers (such as the optical fiber illustrated in FIG. 1) in accordance with at least one example embodiment.

FIG. 3 is a flowchart illustrating an example method for preparing an optical fiber (such as the optical fiber illustrated in FIG. 1) in accordance with at least one example embodiment.

FIG. 4 is a flowchart illustrating an example method of using an optical fiber (such as the optical fiber illustrated in FIG. 1) in accordance with at least one example embodiment.

FIG. 5A is a graphical demonstrating illustrating extension coefficients for optical fibers (such as the optical fiber illustrated in FIG. 1 and/or optical fiber prepared in accordance with the method illustrated FIG. 3) including nanoparticles having different sizes.

FIG. 5B is a graphical demonstration illustrating normalized axial light intensity as a function of depth (z) along optical fibers (such as the optical fibers illustrated in FIG. 1 and/or optical fiber prepared in accordance with the method illustrated FIG. 3) including nanoparticles having different sizes.

FIG. 5C is a graphical demonstration illustrating average light intensity in surrounding medium including photosynthetic microorganisms as a function of depth (z) for optical fibers (such as the optical fiber illustrated in FIG. 1 and/or optical fiber prepared in accordance with the method illustrated FIG. 3) including nanoparticles having different sizes.

FIG. 6A is a graphical demonstration illustrating extinction coefficients for an optical fibers (such as the optical fiber illustrated in FIG. 1 and/or optical fiber prepared in accordance with the method illustrated FIG. 3) including nanoparticles having a size distribution of silica nanoparticles of 490 nanometers as a function of silica weight fraction.

FIG. 6B is a graphical demonstration illustrating normalized emission intensity from the side and end of optical fibers (such as the optical fiber illustrated in FIG. 1 and/or optical fiber prepared in accordance with the method illustrated FIG. 3) as a function of nanoparticle weight fraction.

FIG. 7A is a graphical demonstration illustrating growth curves for algal cultures under different fiber conditions, including one condition including an optical fiber such as the optical fiber illustrated in FIG. 1 and/or an optical fiber prepared in accordance with the method illustrated FIG. 3, where the shaded region indicates fiber-integrated period.

FIG. 7B is a graphical demonstration illustrating volumetric biomass productivities for the different fiber conditions of FIG. 7A.

FIG. 7C is a graphical demonstration illustrating photosynthetic efficiency for the different fiber conditions of FIG. 7A.

FIG. 8A is a graphical demonstration illustrating structural stability of optical fibers (such as the optical fiber illustrated in FIG. 1 and/or optical fiber prepared in accordance with the method illustrated FIG. 3) over sixty days in an algal culture.

FIG. 8B is a graphical demonstration of optical transmittance for different optical materials, including one example an optical fiber such as the optical fiber illustrated in FIG. 1 and/or an optical fiber prepared in accordance with the method illustrated FIG. 3, over sixty days in an algal culture.

FIG. 9 is a graphical demonstration illustrating sixty-day semi-continuous cultivation measurements of different hydrogels, including one example including an optical fiber such as the optical fiber illustrated in FIG. 1 and/or an optical fiber prepared in accordance with the method illustrated FIG. 3.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

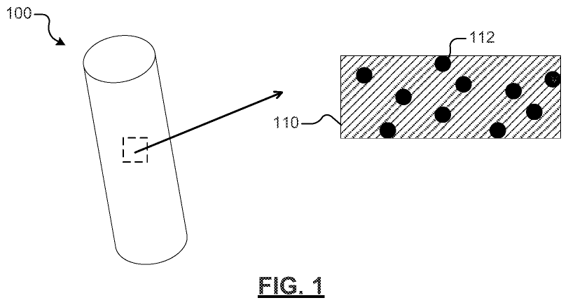

Passive light redistribution strategies that use index-matched, bulk scattering optical fibers to overcome light attenuation and enhance biomass productivity in dense cultures of photosynthetic microorganisms are provided. For example, FIG. 1 is an illustration of an example optical fiber 100 in accordance with at least one example embodiment.

The optical fiber 100 may include (or may be defined by) a hydrogel matrix 110. The hydrogel matrix 110 may be a three-dimensional network of hydrophilic polymers that may be shaped to take a variety of configurations. For example, in at least one example embodiment, as illustrated, the optical fiber may take a cylindrical shape. Although a cylindrical shape is illustrated, it should be appreciated that, in various other example embodiments, the optical fiber 100 may take a variety of other configurations in line with the expected use of the optical fiber 100.

In at least one example embodiment, the optical fiber may have an average diameter greater than or equal to about 0.4 millimeters (e.g., greater than or equal to about 0.6 millimeters, greater than or equal to about 0.8 millimeters, greater than or equal to about 1 millimeters, greater than or equal to about 1.5 millimeters, greater than or equal to about 2 millimeters, greater than or equal to about 2.5 millimeters, greater than or equal to about 3 millimeters, greater than or equal to about 3.5 millimeters, greater than or equal to about 4 millimeters, or greater than or equal to about 4.5 millimeters). In at least one example embodiment, the optical fiber may have an average diameter less than or equal to about 5 millimeters (e.g., less than or equal to about 4.5 millimeters, less than or equal to about 4 millimeters, less than or equal to about 3.5 millimeters, less than or equal to about 3 millimeters, less than or equal to about 2.5 millimeters, less than or equal to about 2 millimeters, less than or equal to about 1.5 millimeters, less than or equal to about 1 millimeters, less than or equal to about 0.8 millimeters, or less than or equal to about 0.6 millimeters). In at least one example embodiment, the optical fiber may have an average diameter greater than or equal to about 0.4 millimeters to less than or equal to about 5 millimeters.

In at least one example embodiment, the optical fiber may have an average length (or height) greater than or equal to about 40 millimeters (e.g., greater than or equal to about 42 millimeters, greater than or equal to about 44 millimeters, greater than or equal to about 46 millimeters, greater than or equal to about 48 millimeters, greater than or equal to about 50 millimeters, greater than or equal to about 52 millimeters, greater than or equal to about 54 millimeters, greater than or equal to about 56 millimeters, or greater than or equal to about 58 millimeters). In at least one example embodiment, the optical fiber may have an average length (or height) less than or equal to about 60 millimeters (e.g., less than or equal to about 58 millimeters, less than or equal to about 56 millimeters, less than or equal to about 54 millimeters, less than or equal to about 52 millimeters, less than or equal to about 50 millimeters, less than or equal to about 48 millimeters, less than or equal to about 46 millimeters, less than or equal to about 44 millimeters, or less than or equal to about 42 millimeters). In at least one example embodiment, the optical fiber may have an average length (or height) greater than or equal to about 40 millimeters to less than or equal to about 60 millimeters.

The hydrophilic polymers may be selected to achieve a close refractive-index match between the hydrogel matrix 110 and an intended photosynthetic microorganisms to help facilitate efficient lateral light extraction. For example, in at least one example embodiment, the hydrogel matrix 110 may exhibit a refractive index of about 1.341. In at least one example embodiment, the hydrophilic polymers may include polyvinyl alcohol, poly(ethylene glycol), polyacrylamide, poly(acrylic acid), alginate, gelatin, chitosan, dextran, hyaluronic acid, cellulose derivatives (e.g., hydroxyethyl cellulose, hydroxypropyl cellulose, hydroxypropyl methylcellulose), or any combination thereof. For example, the hydrophilic polymers may be selected from the group consisting of: polyvinyl alcohol, poly(ethylene glycol), polyacrylamide, poly(acrylic acid), alginate, gelatin, chitosan, dextran, hyaluronic acid, hydroxyethyl cellulose, hydroxypropyl cellulose, hydroxypropyl methylcellulose, and combinations thereof.

In at least one example embodiment, the hydrophilic polymers may be crosslinked to define the hydrogel matrix 110 using a crosslinker, such as glutaraldehyde (GA), vinyl-based crosslinkers (such as N,N′-methylenebisacrylamide (MBAA), polyethylene glycol diacrylate (PEGDA), ethylene glycol dimethacrylate (EGDMA), divinyl sulfone), epichlorohydrin, or any combination thereof. For example, the crosslinker may be selected from the group consisting of: glutaraldehyde (GA), N,N′-methylenebisacrylamide (MBAA), polyethylene glycol diacrylate (PEGDA), ethylene glycol dimethacrylate (EGDMA), divinyl sulfone, epichlorohydrin, or any combination thereof. In at least one example embodiment, a weight ratio of the crosslinker to the hydrophilic polymers may be greater than or equal to about 1:100 and less than or equal to about 1:10.

The optical fiber 100 may further include a plurality of nanoparticles 120 embedded within the hydrogel matrix 110. In at least one example embodiment, the nanoparticles 120 may be uniformly distributed in the hydrogel matrix 110. The nanoparticles 120 may act as internal scattering centers that can help to redirect photons from axial penetration to lateral emission. The nanoparticles may be non-absorbing in the photosynthetically active radiation spectrum, such that scattering is purely elastic with negligible photon energy loss from light absorption. Accordingly, using the hydrogel matrix 110 and the nanoparticles 120 in combination may help to leverage two synergistic light redistribution mechanisms: (1) interfacial extraction enabled by refractive index matching between the hydrogel matrix and the photosynthetic medium, and (2) bulk scattering induced by nanoparticles.

The volume and weight fractions of the nanoparticles 120 relative to the hydrogel matrix 110 may be adjusted and tuned based on the intended use, including, for example, type of photosynthetic microorganisms and/or environmental factors. In at least one example embodiment, the optical fiber 100 may have a weight fraction of the nanoparticles 120 in the hydrogel matrix 110 that is greater than 0 percent (e.g., greater than about 0.05 percent, greater than about 0.1 percent, greater than about 0.15 percent, greater than about 0.2 percent, greater than about 0.25 percent, greater than about 0.3 percent, or greater than about 0.35 percent). In at least one example embodiment, the optical fiber 100 may have a weight fraction of the nanoparticles 120 in the hydrogel matrix 110 that is less than or equal to about 0.36 percent (e.g., less than or equal to about 0.35 percent, less than or equal to about 0.3 percent, less than or equal to about 0.25 percent, less than or equal to about 0.2 percent, less than or equal to about 0.15 percent, less than or equal to about 0.1 percent, or less than or equal to about 0.05 percent). In at least one example embodiment, the optical fiber 100 may have a weight fraction of the nanoparticles 120 in the hydrogel matrix 110 that is greater than 0 percent to less than or equal to about 0.36 percent.

The nanoparticles 120 may include, for example, high-index nanoparticles that have refractive indexes greater than that of the surrounding hydrogel matrix 110 In at least one example embodiment, the nanoparticles 120 may include silica (SiO₂), zirconium dioxide (ZrO₂), titanium dioxide (TiO₂), zinc oxide (ZnO), aluminum oxide (Al₂O₃), or any combination thereof. Silica (SiO₂), for example, has a refractive index of about 1.46.

In at least one example embodiment, the nanoparticles 120 may have a particle distribution greater than or equal to about 200 nanometers (e.g., greater than or equal to about 300 nanometers, greater than or equal to about 400 nanometers, greater than or equal to about 500 nanometers, greater than or equal to about 600 nanometers, greater than or equal to about 700 nanometers, greater than or equal to about 800 nanometers, greater than or equal to about 900 nanometers, greater than or equal to about 1,000 nanometers, greater than or equal to about 1,100 nanometers, greater than or equal to about 1,200 nanometers, greater than or equal to about 1,300 nanometers, greater than or equal to about 1,400 nanometers, greater than or equal to about 1,500 nanometers, greater than or equal to about 1,600 nanometers, or greater than or equal to about 1,700 nanometers). In at least one example embodiment, the nanoparticles 120 may have a particle distribution less than or equal to about 1,800 nanometers (e.g., less than or equal to about 1,700 nanometers, less than or equal to about 1,600 nanometers, less than or equal to about 1,500 nanometers, less than or equal to about 1,400 nanometers, less than or equal to about 1,300 nanometers, less than or equal to about 1,200 nanometers, less than or equal to about 1,100 nanometers, less than or equal to about 1,000 nanometers, less than or equal to about 900 nanometers, less than or equal to about 800 nanometers, less than or equal to about 700 nanometers, less than or equal to about 600 nanometers, less than or equal to about 500 nanometers, less than or equal to about 400 nanometers, or less than or equal to about 300 nanometers). In at least one example embodiment, the nanoparticles 120 may have a particle distribution greater than or equal to about 200 nanometers to less than or equal to about 1,800 nanometers.

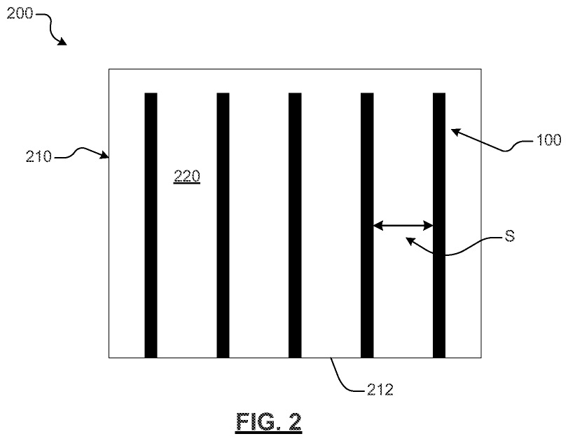

In various aspects, photobioreactors including optical fibers (such as the optical fiber illustrated in FIG. 1) are provided. For example, FIG. 2 is a sideview of an example photobioreactor 200 including one or more optical fibers 100 in accordance with at least one example embodiment. The photobioreactor 200 may include a housing 210 that defines a cavity 220, where the cavity 220 is configured for receiving a photosynthetic medium and the one or more optical fibers 100. In at least one example embodiment, the one or more optical fibers 100 may be supported by the housing 210. For example, as illustrated, the one or more optical fibers 10 may be provided as distinct columns that extend from a bottom structure 212 of the housing 210. Although not illustrated, it should be appreciated that, in various other example embodiments, at least one of the one or more optical fibers 100 may be supported by the bottom structure 112 and one or more other structures also defining the housing 210 and/or by at least one of the one or more other structures defining the housing 210 and/or supported by an exterior structure and free floating within the cavity 220.

In at least one example embodiment, the one or more optical fibers 100 may collectively occupy greater than or equal to about 0.02 percent (e.g., greater than or equal to about 0.05 percent, greater than or equal to about 0.1 percent, greater than or equal to about 0.2 percent, greater than or equal to about 0.4 percent, greater than or equal to about 0.6 percent, greater than or equal to about 0.8 percent, greater than or equal to about 1 percent, greater than or equal to about 1.2 percent, greater than or equal to about 1.4 percent, greater than or equal to about 1.6 percent, greater than or equal to about 1.8 percent, greater than or equal to about 2 percent, greater than or equal to about 2.2 percent, greater than or equal to about 2.4 percent, greater than or equal to about 2.6 percent, or greater than or equal to about 2.8 percent) of a total volume of the cavity 220. In at least one example embodiment, the one or more optical fibers 100 may collectively occupy less than or equal to about 3 percent (e.g., less than or equal to about 2.8 percent, less than or equal to about 2.6 percent, less than or equal to about 2.4 percent, less than or equal to about 2.2 percent, less than or equal to about 2 percent, less than or equal to about 1.8 percent, less than or equal to about 1.6 percent, less than or equal to about 1.4 percent, less than or equal to about 1.2 percent, less than or equal to about 1 percent, less than or equal to about 0.8 percent, less than or equal to about 0.6 percent, less than or equal to about 0.4 percent, less than or equal to about 0.2 percent, less than or equal to about 0.1 percent, or less than or equal to about 0.05 percent) of a total volume of the cavity 220. In at least one example embodiment, the one or more optical fibers 100 may collectively occupy greater than or equal to about 0.02 percent to less than or equal to about 3 percent of a total volume of the cavity 220.

In at least one example embodiment, a spacing(S) between a first optical fiber of the one or more optical fibers 100 and a second optical fiber of the one or more optical fibers 100 may be greater than or equal to about 10 millimeters (e.g., greater than or equal to about 11 millimeters, greater than or equal to about 12 millimeters, greater than or equal to about 13 millimeters, greater than or equal to about 14 millimeters, greater than or equal to about 15 millimeters, greater than or equal to about 16 millimeters, greater than or equal to about 17 millimeters, greater than or equal to about 18 millimeters, or greater than or equal to about 19 millimeters). In at least one example embodiment, a spacing(S) between a first optical fiber of the one or more optical fibers 100 and a second optical fiber of the one or more optical fibers 100 may be less than or equal to about 20 millimeters (e.g., less than or equal to about 19 millimeters, less than or equal to about 18 millimeters, less than or equal to about 17 millimeters, less than or equal to about 16 millimeters, less than or equal to about 15 millimeters, less than or equal to about 14 millimeters, less than or equal to about 13 millimeters, less than or equal to about 12 millimeters, or less than or equal to about 11 millimeters). In at least one example embodiment, a spacing(S) between a first optical fiber of the one or more optical fibers 100 and a second optical fiber of the one or more optical fibers 100 may be greater than or equal to about 10 millimeters to less than or equal to about 20 millimeters.

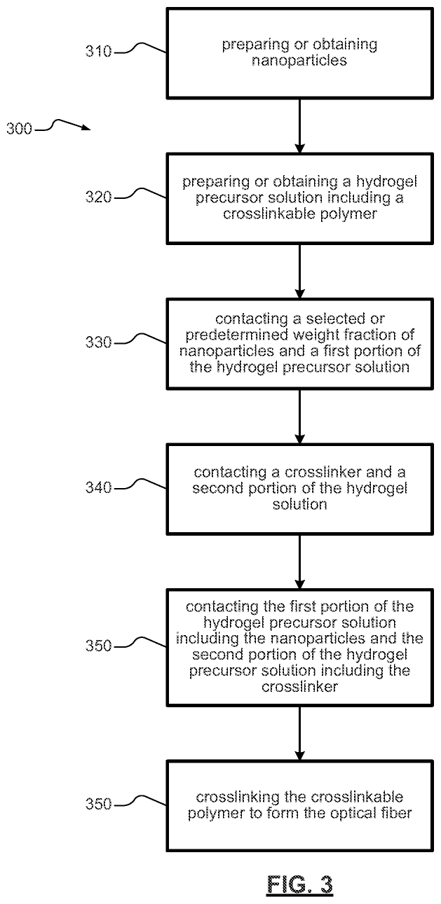

In various aspects, methods for preparing an optical fiber defined by a polyvinyl alcohol-silica hydrogel (like the optical fiber 100 illustrated in FIG. 1) are provided. For example, FIG. 3 is a flowchart illustrating an example method 300 for preparing an optical fiber (such as the optical fiber illustrated in FIG. 1) in accordance with at least one example embodiment.

The method 300 may include contacting 330 a selected or predetermined weight fraction of the nanoparticles (e.g., nanoparticles 120) and a first portion of a hydrogel precursor solution. In at least one example embodiment, the selected or predetermined nanoparticle weight fraction may be greater than 0 weight to less than or equal to about 0.36 weight percent. In at least one example embodiment, the contacting 330 may include adding the nanoparticles to the first portion of hydrogel precursor solution. In at least one example embodiment, the contacting 330 may include applying a mixing force, for example, using a centrifugal mixer, to homogenize the nanoparticles within the hydrogel precursor solution. In at least one example embodiment, the contacting 330 may also include one or more de-gassings, including, for example, after applying the mixing force.

In at least one example embodiment, the method 300 may include preparing or obtaining 310 the nanoparticles. The nanoparticles may be prepared or obtained 310 using a modified Stöber method. In at least one example embodiment, the method 300 may include preparing or obtaining 320 the hydrogel precursor solution. The hydrogel precursor solution may be prepared or obtained 320 by contacting dissolving a crosslinkable polymer (e.g., polyvinyl alcohol), for example, in powder form, in water and heating the mixture to a predetermined or selected temperature (e.g., about 90° C.) for a predetermined or selected period of time (e.g., about 4 hours) until an optically clear solution is obtained. Although the preparing or obtaining 310 of the nanoparticles is illustrated as occurring before the preparing or obtaining 320 of the hydrogel precursor solution, it should be appreciated that, in various example embodiments, the preparing or obtaining 320 of the hydrogel precursor solution may occur before or concurrent with (at least in part) the preparing or obtaining 310 the nanoparticles.

The method 300 may include contacting 340 a crosslinker (e.g., glutaraldehyde) and a second portion of the hydrogel precursor solution, where the second portion of the hydrogel precursor solution is different from the first portion of the hydrogel precursor solution. In at least one example embodiment, the contacting 340 may include adding the crosslinker to the second portion of the hydrogel precursor solution. In at least one example embodiment, the contacting 340 may include applying a mixing force. In at least one example embodiment, the contacting 340 may include one or more de-gassings, including, for example, after the application of the mixing force. Although the contacting 340 of the crosslinker to the second portion of the hydrogel precursor solution is illustrated as following the contacting 330 of the nanoparticles to the first portion of the hydrogel precursor solution, it should be appreciated that, in various example embodiments, the contacting 340 of the crosslinker to the second portion of the hydrogel precursor solution may occur before or concurrent (at least in part) with the contacting 330 of the nanoparticles to the first portion of the hydrogel precursor solution.

The method 300 may include contacting 350 the first portion of the hydrogel precursor solution including the nanoparticles with the second portion of the hydrogel precursor solution including the crosslinkers. In at least one example embodiment, a volume ratio of the first portion of the hydrogel precursor solution including the nanoparticles to the second portion of the hydrogel precursor solution including the crosslinkers may be about 1:1. In at least one example embodiment, the contacting 350 may include applying a mixing force. In at least one example embodiment, the contacting 350 may include one or more de-gassings, including, for example, after the application of the mixing force.

The method 300 may include, after the contacting 350, crosslinking 360 the crosslinkable polymer (e.g., polyvinyl alcohol) in the combined first and second portions of the hydrogel precursor solution for a predetermined or selected period of time (e.g., about 4 hours) to form the optical fiber. Although not illustrated, it should be appreciated that, in various example embodiments, the method 300 may include, before or during the crosslinking 260, moving the of the hydrogel precursor solution to a mold (e.g., polyvinyl chloride tube) to form a predetermined shape of the optical fibers. In at least one example embodiments, the prepared optical fibers may be extruded from the mold by swelling the mold in dichloromethane and rinsing thoroughly. Further, although not illustrated, it should be appreciated that, in various example embodiments, the method 300 may include placing the as-prepared optical fibers in water for storage.

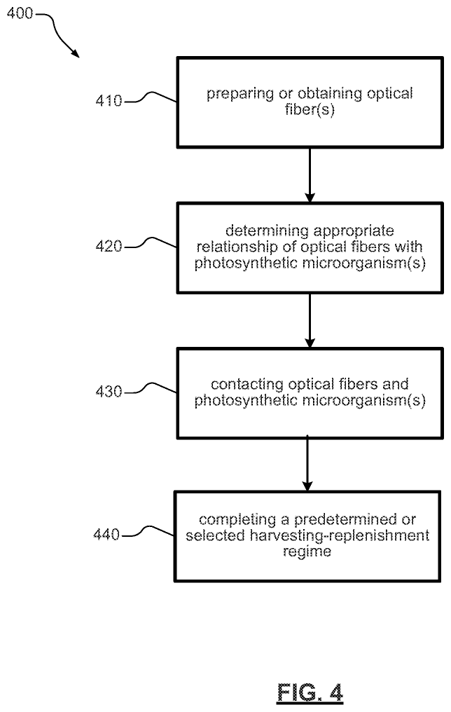

In various aspects, methods for using an optical fiber defined by a polyvinyl alcohol-silica hydrogel (like the optical fiber 100 illustrated in FIG. 1) are provided. For example, FIG. 4 is a flowchart illustrating an example method 400 of using an optical fiber (such as the optical fiber illustrated in FIG. 1) in accordance with at least one example embodiment.

The method 400 may include contacting 430 an optical fiber and photosynthetic microorganism (e.g., algal culture), for example, in a photobioreactor (see, e.g., photobioreactor 200 illustrated in FIG. 2), and completing 440 a predetermined or selected harvest-replenishment regime. In at least one example embodiment, the harvest-replenishment regime may include, for example, growing the photosynthetic microorganism under illumination, harvesting a portion of the culture, and adding fresh culture media to restore the culture volume. The process may be repeated in a continuous or semi-continuous manner. Although not illustrated, it should be appreciated that, in various example embodiments, the method 400 may include determining or selecting the harvest-replenishment regime.

In at least one example embodiment, the method 400 may include determining 420 an appropriate relationship between the optical fiber and the photosynthetic microorganism. The relationship between the optical fiber and the photosynthetic microorganism may depend on the depth (or height) of the photosynthetic microorganism, as well as cell density, and determining 420 the appropriate relationship between the optical fiber and the photosynthetic microorganism may include determining the depth and/or cell density of the photosynthetic microorganism.

In at least one example embodiment, an appropriate relationship between the optical fiber and the photosynthetic microorganism may include greater than or equal to about 450 square centimeters (e.g., greater than or equal to about 500 square centimeters, greater than or equal to about 550 square centimeters, greater than or equal to about 600 square centimeters, greater than or equal to about 650 square centimeters, greater than or equal to about 700 square centimeters, greater than or equal to about 750 square centimeters, greater than or equal to about 800 square centimeters, greater than or equal to about 850 square centimeters, or greater than or equal to about 900 square centimeters) of the optical fiber surface area per about 1 gram of the photosynthetic microorganism. In at least one example embodiment, an appropriate relationship between the optical fiber and the photosynthetic microorganism may include less than or equal to about 950 square centimeters (e.g., less than or equal to about 900 square centimeters, less than or equal to about 850 square centimeters, less than or equal to about 800 square centimeters, less than or equal to about 750 square centimeters, less than or equal to about 700 square centimeters, less than or equal to about 650 square centimeters, less than or equal to about 600 square centimeters, less than or equal to about 550 square centimeters, or less than or equal to about 500 square centimeters) of the optical fiber surface area per about 1 gram of the photosynthetic microorganism. In at least one example embodiment, an appropriate relationship between the optical fiber and the photosynthetic microorganism may include greater than or equal to about 450 square centimeters to less than or equal to about 950 square centimeters of the optical fiber surface area per about 1 gram of the photosynthetic microorganism.

In at least one example embodiment, the method 400 may include preparing and/or obtaining 410 the optical fiber (such as, for example, using the method 400 as illustrated in FIG. 3). Although the preparing and/or obtaining 410 the optical fibers is illustrated as occurring after the determining 420 of the appropriate relationship between the optical fiber and the photosynthetic microorganism, it should be appreciated that, in various example embodiments, the determining 420 of the appropriate relationship between the optical fiber and the photosynthetic microorganism may occur before or concurrent (at least in part) with the preparing and/or obtaining 410 the optical fibers.

Certain features of the current technology are further illustrated in the following non-limiting examples.

Example Synthesis of Silica Nanoparticles. Silica nanoparticles were synthesized using a modified Stöber method. A mixture of about 54 milliliters (mL) ethanol and about 7.8 milliliters deionized water was heated to about 50° C. for about 10 minutes, followed by the addition of about 3.4 milliliters tetraethyl orthosilicate (TEOS) and stirring for about 20 minutes to ensure uniform dispersion. To initiate the reaction, about 0.6 milliliters of 0.40 M sodium hydroxide (NaOH) solution was added dropwise, and the reaction was allowed to proceed at about 50° C. for about 20 hours, yielding silica nanoparticles with a diameter of about 493 nanometers (nm). To synthesize nanoparticles with different sizes, the volume of sodium hydroxide (NaOH) was adjusted to about 1 milliliter for larger particles (about 1741 nanometers) and 0.1 milliliter for smaller particles (about 212 nanometers). The nanoparticles were isolated by centrifugation at about 6,000 rotations per minute (rpm) for 10 minutes and washed three times with deionized water. To remove any residual sodium ions, the nanoparticles were refluxed in a 4% hydrochloric acid solution for about 3 hours, then washed and neutralized with deionized water until the solution reached neutral pH. The prepared silica nanoparticles were dried overnight at about 80° C.

Example Synthesis Process for Optical Fibers. To prepare a 10 weight percent polyvinyl alcohol solution, 10 grams (g) of polyvinyl alcohol powder was dissolved in about 90 milliliters of deionized water and heated at about 90° C. for about 4 hours until a clear solution was obtained. For the polyvinyl alcohol-silica hydrogel, about 80 microliters (μL) of 37 weight percent hydrochloric acid and a predetermined weight fraction (e.g., 0 weight percent to about 0.36 weight percent) and particle size (e.g., 212 nanometers, 493 nanometers, or 1741 nanometers) of silica nanoparticles were added to 10 grams of the polyvinyl alcohol solution, homogenized using a centrifugal mixer (AR-100, THINKY USA) for about 90 seconds, and degassed for about 30 seconds. Meanwhile, 80 microliters of glutraraldehyde was added to another about 10 grams of the polyvinyl alcohol solution, followed by about 90 second mixing and 30 second degassing. The two solutions were then combined in a 1:1 ratio by 90 second mixing and 30 second degassing. To fabricate scattering optical fibers, the resultant precursor solution was infused into a polyvinyl chloride (PVC) tubing (inner diameter: about 2.4 millimeters (mm)) and allowed to crosslink at room temperature for 4 hours. The scattering optical fibers were extruded by swelling the tubing in dichloromethane, rinsed thoroughly, and stored in deionized water for subsequent use. To fabricate scattering membranes, a thin-film mold was prepared by placing about 0.5-millimeter-thick spacers between two glass slides. The precursor solution was poured into the mold and crosslinked at room temperature for about 4 hours to form a about 0.5-millimeter-thick polyvinyl alcohol-silica hydrogel membrane. The hydrogel membranes were then carefully rinsed and stored in deionized water.

Particle Size Characterization. To investigate the size effect, three silica populations with mean diameters of 212 nanometers, 493 nanometers, and 1741 nanometers were synthesized by adjusting the pH during their condensation. These sizes span below, near, and above the optical wavelength, respectively, while a constant silica weight fraction of 0.06% was maintained in the silica-containing polyvinyl alcohol hydrogels. Although the total transmittance of these silica-containing polyvinyl alcohol hydrogel samples remained almost constant across all particle diameters, larger silica nanoparticles resulted in reduced direct transmittance and enhanced diffuse transmittance.

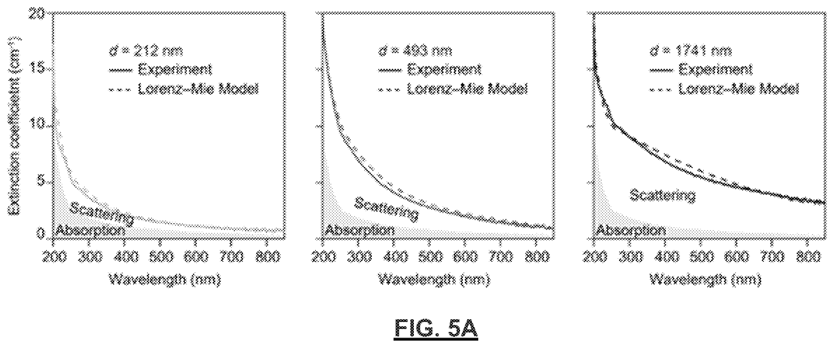

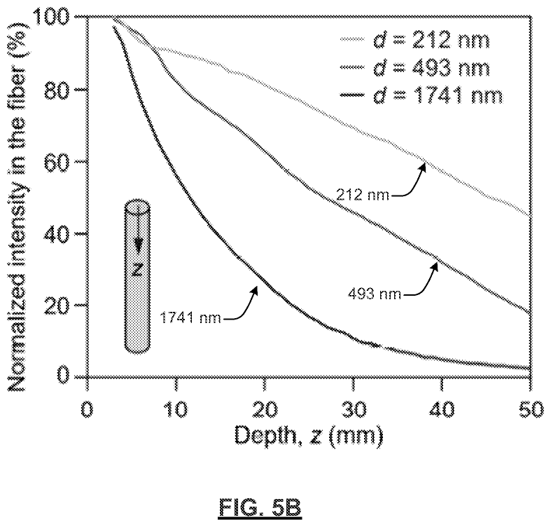

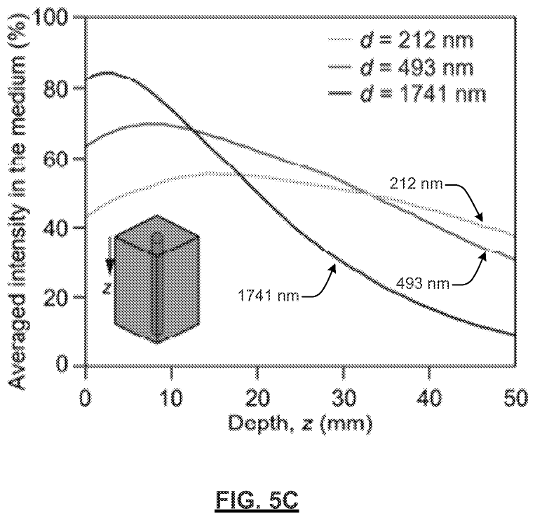

Using the Beer-Lambert law, extinction coefficients were extracted from experimental direct transmittance measurements. To better understand the scattering mechanisms, Lorenz-Mie theory was applied to decompose the measured extinction coefficients into silica-induced scattering and hydrogel-related absorption. This optical model accounted for the polydisperse particle size distribution and the wavelength-dependent refractive indices of silica and water, enabling realistic predictions of optical behaviors. The theoretically calculated extinction spectra showed good agreement with experimental results across all particle sizes (e.g., of 212 nanometers, 493 nanometers, and 1741 nanometers) and wavelengths (e.g., 200 nanometers to 850 nanometers). The absorption coefficients remained constant regardless of particle size, indicating that absorption is dominated by the polyvinyl alcohol hydrogel alone. In contrast, the scattering coefficients varied significantly with particle size, particularly in the visible spectrum (e.g., 400 nanometers to 700 nanometers). For example, hydrogels with 1741 nanometer silica nanoparticles exhibited a scattering coefficient at about 490 nanometers that was about 7.4-fold larger than that of 212-nanometer particles. FIG. 5A is a graphical demonstration illustrating extension coefficients for optical fibers (such as the optical fiber illustrated in FIG. 1 and/or optical fiber prepared in accordance with the method illustrated FIG. 3) including nanoparticles having different sizes.

To visualize light redistribution, optical fibers with varied particle sizes were fabricated and imaged to obtain their emission patterns under 490 nanometers LED illumination. FIG. 5B is a graphical demonstration illustrating normalized axial light intensity as a function of depth (z) along optical fibers (such as the optical fiber illustrated in FIG. 1 and/or optical fiber prepared in accordance with the method illustrated FIG. 3) including nanoparticles having different sizes. The graphical demonstration of FIG. 5B shows diameter-dependent optical attenuation. FIG. 5C is a graphical demonstration illustrating average light intensity in surrounding medium including photosynthetic microorganisms as a function of depth (z) for optical fibers (such as the optical fiber illustrated in FIG. 1 and/or optical fiber prepared in accordance with the method illustrated FIG. 3) including nanoparticles having different sizes.

The size-dependent scattering manifests in fiber performance: larger nanoparticles yield stronger side emission but shorter axial penetration due to the increased scattering effect. Specifically, fibers with 1741 nanometer silica nanoparticles showed a short penetration depth of 1.6 centimeters (cm), whereas those with 212 nanometer silica nano particles achieved deeper axial propagation of 6.2 centimeters, albeit with weaker lateral redistribution. The 493 nanometer silica nanoparticles offered a balanced profile between side emission and penetration depth, making them suitable for typical algal reactors with 4.0-centimeter characteristic depth.

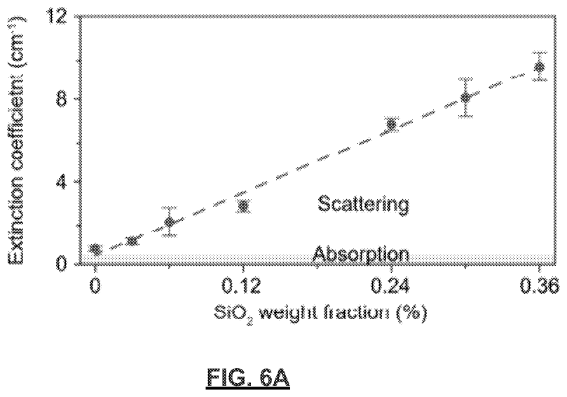

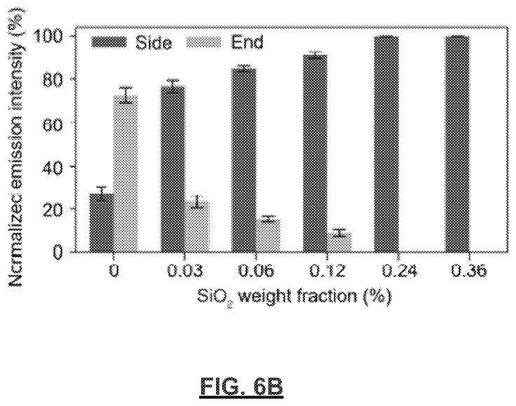

Nanoparticle Concentration Characterization. The impact of silica nanoparticle concentration was evaluated by varying their weight fraction from 0% to 0.36%. Increasing silica content caused a pronounced drop in direct transmittance from 97% to 62% at 490 nanometers due to enhanced scattering. The derived extinction coefficients and the scattering component increase linearly with silica weight fraction, while the absorption component remained unchanged. FIG. 6A is a graphical demonstration illustrating extinction coefficients for an optical fiber (such as the optical fiber illustrated in FIG. 1 and/or optical fiber prepared in accordance with the method illustrated FIG. 3) including nanoparticles having a size distribution of silica nanoparticles of 490 nanometers as a function of silica weight fraction. The graphical demonstration of FIG. 6A shows constant absorption but increasing scattering with nanoparticle weight fraction.

Side-view images of fibers revealed that as silica content increased, side emission became brighter and more uniform, while end emission weakened significantly. Notably, the bright ring-shaped regions in the end-view imaging represent a hollow glass tubing used to enclose the fibers, where side-emitted light becomes trapped along the tubing walls by total internal reflection. Quantitative analysis further confirmed this transition from end-dominated to side emission dominated as silica loading increased and fibers without silica nanoparticles (0%) transmitted most light to the distal end, whereas fibers containing greater than or equal to about 0.12% silica nanoparticles exhibited strong side emission with minimal end loss. FIG. 6B is a graphical demonstration illustrating normalized emission intensity from the side and end of optical fibers (such as the optical fiber illustrated in FIG. 1 and/or optical fiber prepared in accordance with the method illustrated FIG. 3) as a function of nanoparticle weight fraction. The graphical demonstration of FIG. 6B shows a transition from end-dominated to side-dominated emission.

Effects of Fiber Geometry. Changing the fiber diameter had little effect on axial attenuation since all fibers shared identical optical properties, but smaller diameters increased lateral emission by promoting more frequent light extraction at the fiber-medium interface. In contrast, inter-fiber spacing strongly affected illumination uniformity: large spacing produced dark regions between fibers, whereas reducing the spacing progressively merged the emission zones and generated a uniform light field throughout the depth.

Opto-Biological Modeling Framework. To explore the mechanistic links between fiber development, light distribution and algal productivity, a coupled opto-biological modeling framework that simulates spatial light field and biomass production in algal reactors with or without fibers was established. The framework included three sequential steps, including (1) Lorenz-Mie theory to compute the optical parameters of polyvinyl alcohol-silica hydrogel, (2) Monte-Carlo ray-tracing simulation to generate the three-dimensional absorbed photon number density within a single-fiber unit cell, and (3) an experimentally calibrated algal growth model that converts the local absorbed photon number density into spatially resolved local productivity and the corresponding volumetric average.

Using this framework, the light-dependence of algal cultures was captured: increases in reactor depth or cell density initially enhance productivity but soon plateau or decline when light becomes limiting, which highlights the need to redistribute light within dense and deep algal cultures. Moreover, the optical simulation successfully reproduced the experimentally observed enhancement in light intensity surrounding the fibers, and the coupled algal growth model showed this localized light increase reactivates photosynthesis in deeper regions, thereby elevating the overall volumetric productivity. To identify optimal material designs for scattering hydrogel fibers, the predicted light distribution and resulting biomass product across different fiber compositions were examined. The framework revealed non-monotonic dependencies of volumetric biomass productivity on both silica nanoparticle size and weight fraction. Too small or too dilute particles scatter weakly and fail to redistribute light effectively, whereas very large or highly loaded particles increase scattering anisotropy and forward attenuation, causing excessive scattering losses before light reaches deeper regions. These trends establish an example intermediate optimal regime for maximizing reactor-scale productivity.

Biomass Production in Light-Limited Algal Cultures. C. sorokiniana, a freshwater green microalga known for its scalability and fast growth kinetics was used. The cultures were illuminated with a 100 W·m⁻² blue/red LED array matched to its absorbance spectrum. To systematically assess fiber performance, a high-throughput, bench-scale platform consisting of 50 milliliters photobioreactors mounted on a shaker was prepared. The algae grew photoautotrophically in the carbon-free culture medium and relied solely on carbon dioxide (CO₂) supplied from the atmosphere through air tubing. Because light was rapidly attenuated, particularly in dense cultures, an array of three 6-centimeter long optical fibers were introduced into each 50-milliliter reactor. As the shallow surface layer receives excessive direct illumination, condenser lenses were placed between the LED source and the fibers to focus the incident light into the fibers. To further reduce excess light and prevent photoinhibition at the culture surface, short glass fibers were used as transmitting segments to guide light through the top layer without lateral emission. These transmitting segments were then connected to scattering hydrogel fibers via rubber sleeves. Below the penetration depth, the scattering fibers were arranged in parallel to redistribute light throughout deeper regions of the culture.

Side-view images demonstrate that transmitting fibers alone deliver light to localized distal positions, whereas scattering fibers redistribute light laterally along their length, generating a more uniform illumination profile throughout the culture volume. To quantify algal growth under different light modulation conditions, the optical density at 750 nanometers (OD750), a widely used proxy for algal biomass concentration, was monitored. Its use for C. sorokiniana was validated by calibrating OD750 against both dry weight (DW) and ash-free dry weight (AFDW), both showing strong linear correlations. Unlike direct dry weight and ash-free dry weight measurements, OD750 enables convenient, non-destructive real-time biomass monitoring. Under normal cultivation without additional light modulation, C. sorokiniana displayed the three characteristic growth phases: a lag phase with acclimation (day 0-1), an exponential phase with rapid cell division (day 1-4), and a stationary phase driven primarily by light limitation in dense cultures (day 4-9). Once cultures entered the light-limited stationary phase (OD750≈4), the cultures were transferred into 50 milliliter photobioreactors and subjected to diverse light-modulation strategies, including those integrated with hydrogel fibers. OD750 was tracked every 12 hours. To calculate volumetric and areal biomass productivities, photosynthetic efficiency, and carbon dioxide fixation rate for the entire cultivation time, ash-free dry weight of the algal culture before and after cultivation was measured.

Compared with fiber-free controls, hydrogel fibers remarkably improved both biomass productivity and photosynthetic efficiency by 6.9 times. To benchmark the performance of the scattering hydrogel fibers, a comprehensive comparison was conducted across (1) different fiber materials (glass, polymethyl methacrylate (PMMA), and polyvinyl alcohol-silica hydrogel), (2) fiber geometries and surface structures (short vs. long glass fibers, smooth transmitting vs. nanostructured scattering polymethyl methacrylate (PMMA) fibers), (3) fiber placement within the reactor (center-, internal-wall-, and external-wall-mounted), and (4) active illumination strategies (LED strip and LED mat). All conditions were tested under identical cultivation parameters.

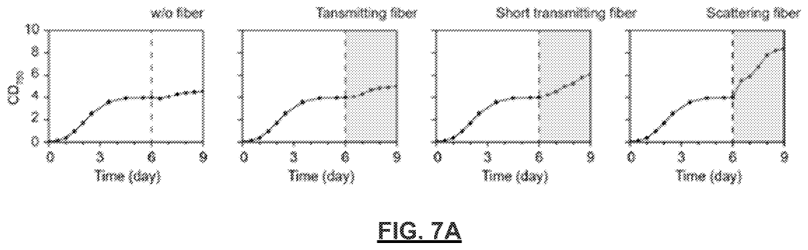

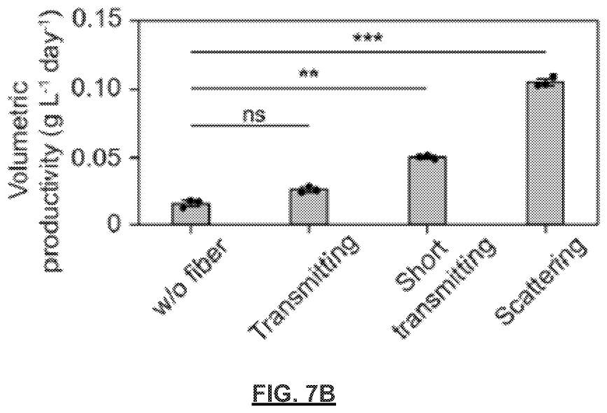

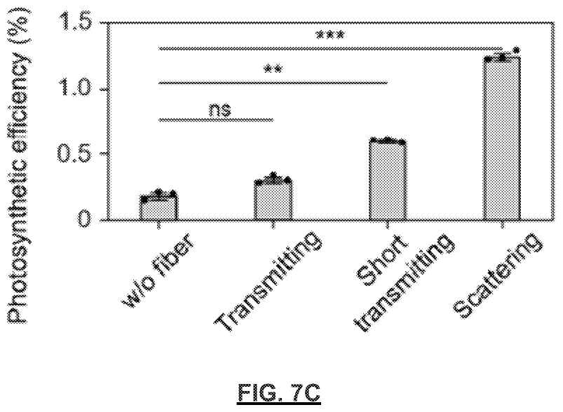

FIG. 7A is a graphical demonstration illustrating growth curves for algal cultures under different fiber conditions, including one condition including an optical fiber such as the optical fiber illustrated in FIG. 1 and/or an optical fiber prepared in accordance with the method illustrated FIG. 3, where the shaded region indicates fiber-integrated period. FIG. 7B is a graphical demonstration illustrating volumetric biomass productivities for the different fiber conditions of FIG. 7A, where statistical significance is represented as follows *p<0.05, **p<0.01, ***p<0.001, and ns=not significant. FIG. 7C is a graphical demonstration illustrating photosynthetic efficiency for the different fiber conditions of FIG. 7A.

Across all passive optical strategies tested, the scattering polyvinyl alcohol-silica hydrogel fibers consistently outperformed other materials: the 6.9 times increase in biomass productivity exceeds the 1.6 to 3.8 times improvements achieved by long or short glass fibers and by transmitting or nanostructured polymethyl methacrylate (PMMA) fibers. Although inserting LED strip or LED mat systems produced comparable productivity gains (e.g., 7.0 to 7.2 times), their high-power demand reduced the photon-to-biomass conversion efficiency to around 0.3%, far below the 1.2% efficiency obtained with passive systems. Further, center-mounted hydrogel fibers were found to yield the strongest improvements in volumetric light redistribution and biomass accumulation relative to wall-mounted configurations. These improvements were also observed in Chlorella pyrenoidosa and Haematococcus pluvialis, where scattering hydrogel fibers (i.e., polyvinyl alcohol-silica hydrogel fibers) increased their growth to similar extents, demonstrating that the fibers function as a universal, species independent light-redistribution tool for algal photosynthesis. Overall, these results reveal that incorporating scattering hydrogel fibers in dense cultures achieve the highest biomass productivity among passive light-delivery systems and match the performance of active LED-based illumination strategies at zero additional energy cost.

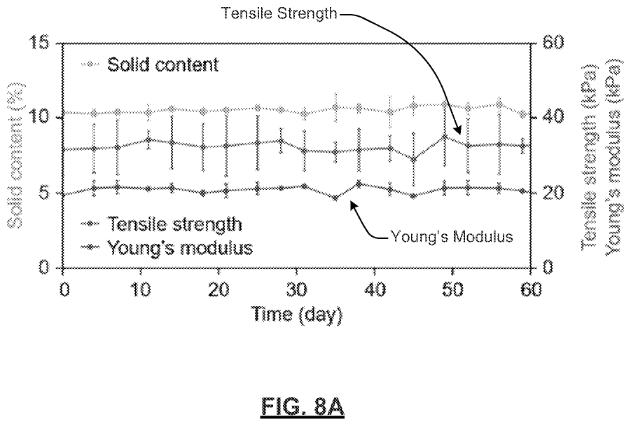

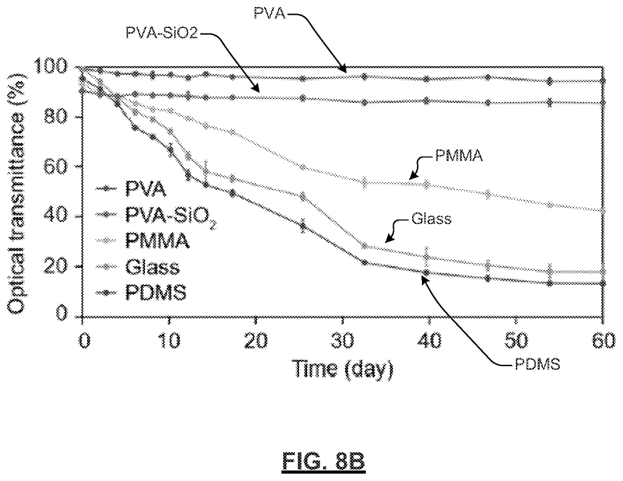

Long-Term Durability of Polyvinyl Alcohol-Silica Hydrogel Fibers. To evaluate the long-term durability of polyvinyl alcohol-silica hydrogel materials, two-month outdoor cultivation experiments under realistic photobioreactor conditions were conducted. Throughout the study, the polyvinyl alcohol-silica hydrogel materials were continuously exposed to natural sunlight, algal medium, fluid agitation, and daily temperature fluctuations. The polyvinyl alcohol-silica hydrogel materials maintained stable mechanical properties during prolonged operation. Uniaxial tensile tests showed that tensile strength (e.g., 32 kPa), Youngs modulus (e.g., 21 kPa), and solid content (10.5%) remained unchanged over sixty days, with no evidence of degradation or fatigue. When subjected to vigorous vortex mixing, the polyvinyl alcohol-silica hydrogel materials attached to the reactor exhibited excellent stability, showing no detectable displacement or fluttering and behaving comparably to rigid glass and polymethyl methacrylate (PMMA) controls. FIG. 8A is a graphical demonstration illustrating structural stability of optical fibers (such as the optical fiber illustrated in FIG. 1 and/or optical fiber prepared in accordance with the method illustrated FIG. 3) over sixty days in an algal culture.

Surface fouling kinetics under outdoor cultivation was examined. Whereas other optical materials such as glass, polymethyl methacrylate (PMMA), and polydimethylsiloxane (PDMS) materials developed substantial biofilm coverage (e.g., 40 percent to 80 percent) over the sixty days, the polyvinyl alcohol-silica hydrogel materials showed only minimal microbial attachment (e.g., 11.7 percent surface coverage). This anti-fouling behavior is attributed to the strong hydration shell formed by the abundant hydroxyl groups in polyvinyl alcohol, which inhibits protein and microorganism adhesion. Such resistance to biofilm formation is essential for preserving long-term optical performance. The optical transmittance of polyvinyl alcohol-silica hydrogel materials also remained stable over sixty days, in sharp contrast to glass, polymethyl methacrylate (PMMA), and polydimethylsiloxane (PDMS), which lost 45 percent to 70 percent transmittance due to fouling. FIG. 8B is a graphical demonstration of optical transmittance for different optical materials, including one example an optical fiber such as the optical fiber illustrated in FIG. 1 and/or an optical fiber prepared in accordance with the method illustrated FIG. 3, over sixty days in an algal culture. Together, the robust mechanical stability, antifouling properties, and preserved optical transmittance indicate that the scattering hydrogel fibers are suitable for long-term deployment in outdoor photobioreactors with minimal maintenance requirements.

Extended Outdoor Photobioreactor Operation. To validate the fibers under extended real-world conditions, a two-month semi-continuous outdoor cultivation using four 1-L photobioreactors was conducted. Two reactors were equipped with scattering hydrogel fibers, and two identical reactors without fibers served as controls. All reactors began with the same inoculum at an initial biomass concentration of 0.6 g·L⁻¹, corresponding to a very small light penetration depth of 0.23 millimeters. These reactors were operated under natural sunlight and subjected to identical semi-continuous harvest-replenish cycles, in which 300 milliliters of culture was removed every other day and replaced with fresh carbon-free medium. To ensure sufficient inorganic carbon supply, the cultures were continuously aerated with ambient air (e.g., 3.5 L·min⁻¹, 0.04% CO₂). The culture pH was maintained in a mildly basic range to suppress the non-algal overgrowth. Solar irradiance, culture temperature, pH, and volume were tracked throughout the study.

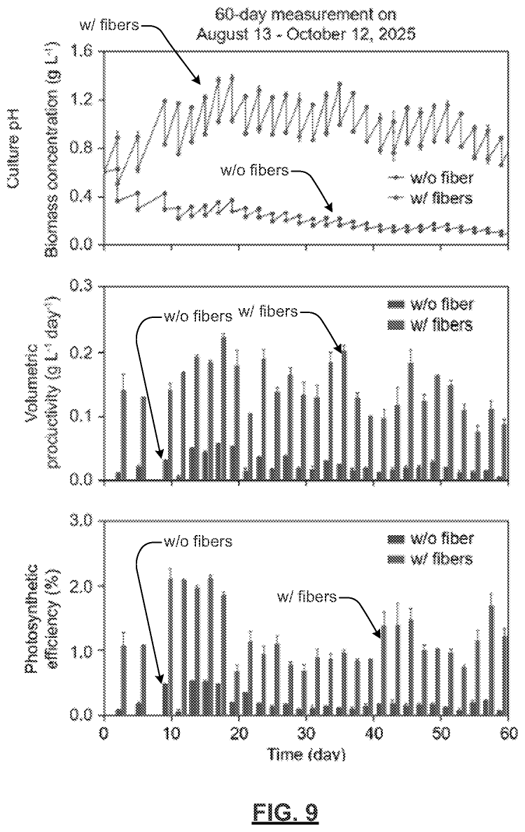

Over the two-month period, solar irradiance and culture temperature gradually decreased as the season transitioned from summer to fall, while pH and volume were maintained the same. Under these fluctuating conditions, the biomass in control reactors without fiber progressively declined to about 0.1 g·L⁻¹. In contrast, fiber-integrated reactors consistently maintained high biomass concentrations of 0.8 to 1.4 g·L⁻¹. FIG. 9 is a graphical demonstration illustrating sixty-day semi-continuous cultivation measurements of different hydrogels, including one example including an optical fiber such as the optical fiber illustrated in FIG. 1 and/or an optical fiber prepared in accordance with the method illustrated FIG. 3. The biomass concentrations were measured every other day after harvesting and volumetric biomass productivity and photosynthetic efficiency were calculated. As illustrated, volumetric productivity, areal productivity, photosynthetic efficiency, and carbon dioxide fixation rate were substantially higher in the fiber-integrated reactors, yielding an average 6.2-fold and a maximum 32.4-fold improvement in biomass productivity. These results match the opto-biological model prediction of a 3.2-fold productivity increase.

Single-day batch growth initiated at a dense concentration (e.g., OD750≈4.6) was also evaluated. Fiber-free reactors exhibited only a 6.1 percent increase in OD750, consistent with severe light attenuation, whereas reactors with fibers achieved a 34.7% increase, corresponding to a 5.7-fold enhancement in biomass production. Finally, solar-tracking experiments showed only minimal improvement relative to static reactors, indicating that light redistribution by the scattering hydrogel fibers is largely insensitive to incident light angle.

Microscopic examination confirmed no morphological differences between cultures grown with and without fibers, and cell diameters maintained around 4 micrometers in both cases. Biochemical analysis showed that biomass harvested from all photobioreactors contained about 75 percent protein, with the remaining fraction consisting of carbohydrates (about 15 percent), lipids (about 5 percent), and minerals (about 5 percent), consistent with the known protein-rich profile of C. sorokiniana. Elemental composition (C/H/N) was also statistically indistinguishable between the two groups. These results indicate that, although the scattering fibers significantly increase biomass productivity and photosynthetic efficiency, they do not alter cell physiology or the biochemical quality of the harvested biomass.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of using an index-matched, bulk-scattering optical fiber during photosynthesis by a photosynthetic microorganism, the method comprising:
contacting the optical fiber and the photosynthetic microorganism, the optical fiber including
a hydrogel matrix, and
a plurality of nanoparticles dispersed in the hydrogel matrix.

2. The method of claim 1, wherein the hydrogel matrix includes a crosslinkable polymer, the crosslinkable polymer including polyvinyl alcohol, poly(ethylene glycol), polyacrylamide, poly(acrylic acid), alginate, gelatin, chitosan, dextran, hyaluronic acid, cellulose derivatives, or any combination thereof.

3. The method of claim 1, wherein the nanoparticles of the plurality of nanoparticles include silica (SiO₂), zirconium dioxide (ZrO₂), titanium dioxide (TiO₂), zinc oxide (ZnO), aluminum oxide (Al₂O₃), or any combination thereof.

4. The method of claim 1, wherein the nanoparticles of the plurality of nanoparticles have a particle distribution greater than or equal to about 200 nanometers to less than or equal to about 1,800 nanometers.

5. The method of claim 1, wherein a weight fraction of the plurality of nanoparticles in the hydrogel matrix ranges from greater than 0 weight percent to less than or equal to about 0.36 weight percent.

6. The method of claim 1, wherein the optical fiber has an average diameter greater than or equal to about 0.4 millimeters to less than or equal to about 5 millimeters.

7. The method of claim 1, wherein a relationship between the optical fiber and the photosynthetic microorganism includes greater than or equal to about 450 square centimeters to less than or equal to about 950 square centimeters of surface area of the optical fiber per about 1 gram of the photosynthetic microorganism.

8. The method of claim 1, wherein the contacting occurs in a photobioreactor, the photobioreactor including a housing that defines cavity configured to receive the photosynthetic microorganism and that supports the optical fiber.

9. The method of claim 8, wherein the optical fiber is a first optical fiber and the photobioreactor further includes a second optical fiber, an average spacing between the first optical fiber and the second optical fiber being greater than or equal to about 10 millimeters to less than or equal to about 20 millimeters.

10. The method of claim 1, further includes
determining a first refractive-index of the photosynthetic microorganism, and
selecting the hydrogel matrix to have a second refractive-index that is the same as the first refractive-index.

11. The method of claim 1, further includes
determining an optimal volumetric ratio of the optical fiber to the photosynthetic microorganism.

12. The method of claim 1, further includes
preparing the hydrogel matrix, the preparing of the hydrogel matrix including contacting a first hydrogel precursor solution including the nanoparticles and a second hydrogel precursor solution including a crosslinker, the first and second hydrogel precursor solutions both including a crosslinkable polymer.

13. An index-matched, bulk scattering optical fiber for use during photosynthesis by a photosynthetic microorganism, the optical fiber comprising:
a hydrogel matrix, and
a plurality of nanoparticles dispersed in the hydrogel matrix, a weight fraction of the nanoparticles in the hydrogel matrix ranging from greater than 0 weight percent to less than or equal to about 0.36 weight percent.

14. The optical fiber of claim 13, wherein
the hydrogel matrix includes a crosslinkable polymer, the crosslinkable polymer including polyvinyl alcohol, poly(ethylene glycol), polyacrylamide, poly(acrylic acid), alginate, gelatin, chitosan, dextran, hyaluronic acid, cellulose derivatives, or any combination thereof; and
the nanoparticles of the plurality of nanoparticles include silica (SiO₂), zirconium dioxide (ZrO₂), titanium dioxide (TiO₂), zinc oxide (ZnO), aluminum oxide (Al₂O₃), or any combination thereof.

15. The optical fiber of claim 13, wherein the nanoparticles of the plurality of nanoparticles have a particle distribution greater than or equal to about 200 nanometers to less than or equal to about 1800 nanometers.

16. A photobioreactor for use during photosynthesis by a photosynthetic microorganism, the photobioreactor comprising:
a housing that defines a cavity, the cavity being configured to receive a photosynthetic microorganism and an optical fiber, the optical fiber including
a hydrogel matrix, and
a plurality of nanoparticles dispersed in the hydrogel matrix.

17. The photobioreactor of claim 16, wherein
the hydrogel matrix includes a crosslinkable polymer, the crosslinkable polymer including polyvinyl alcohol, poly(ethylene glycol), polyacrylamide, poly(acrylic acid), alginate, gelatin, chitosan, dextran, hyaluronic acid, cellulose derivatives, or any combination thereof; and
the nanoparticles of the plurality of nanoparticles include silica (SiO₂), zirconium dioxide (ZrO₂), titanium dioxide (TiO₂), zinc oxide (ZnO), aluminum oxide (Al₂O₃), or any combination thereof.

18. The photobioreactor of claim 16, wherein the optical fiber is a first optical fiber and the photobioreactor further includes one or more other optical fibers that are provided with the first optical fiber as an array of optical fibers.

19. The photobioreactor of claim 18, wherein the array of optical fibers is configured to be received by the cavity and to be at least partially submerged into a bioreactor medium that includes the photosynthetic microorganism.

20. The photobioreactor of claim 18, wherein an average spacing between the first optical fiber and a second optical fiber of the one or more other optical fibers is greater than or equal to about 10 millimeters to less than or equal to about 20 millimeters.
